(19) United States
(12) Patent Application Publication
(10) Pub. No.: US 2024/0165328 A1
(43) Pub. Date: May 23, 2024

(54) ADAPTABLE ASYMMETRIC MEDICAMENT COST COMPONENT IN A CONTROL SYSTEM FOR MEDICAMENT DELIVERY

(71) Applicant: INSULET CORPORATION, Acton, MA (US)

(72) Inventors: Joon Bok LEE, Acton, MA (US); Yibin ZHENG, Hartland, WI (US); Jason O'CONNOR, Acton, MA (US); Trang LY, Concord, MA (US)

(21) Appl. No.: 18/422,986

(22) Filed: Jan. 25, 2024

Related U.S. Application Data
(63) Continuation of application No. 17/330,115, filed on May 25, 2021, now Pat. No. 11,904,140.
(60) Provisional application No. 63/165,252, filed on Mar. 24, 2021.
(60) Provisional application No. 63/158,918, filed on Mar. 10, 2021.

(51) Int. Cl. A61M 5/172; A61B 5/00; A61B 5/145; A61M 5/142; G06Q 30/0283; G16H 20/17

(52) U.S. Cl. CPC A61M 5/1723; A61B 5/14532; A61B 5/4839; A61M 5/142; G06Q 30/0283; G16H 20/17; A61M 2005/14208; A61M 2205/52; A61M 2230/201

(57) ABSTRACT

The exemplary embodiments provide medicament delivery devices that use cost functions in their control systems to determine medicament dosages. The cost function may have a medicament cost component and a performance cost component. The exemplary embodiments may use cost functions having medicament cost components that scale asymmetrically for different ranges of inputs (i.e., different candidate medicament dosages). The variance in scaling for different input ranges provides added flexibility to tailor the medicament cost component to the user and thus provide better management of medicament delivery to the user and better conformance to a performance target. The exemplary embodiments may use a cost function that has a medicament cost component (such as an insulin cost component) of zero for candidate dosages for a range of candidate dosages (e.g., below a reference dosage).

### CROSS-REFERENCE TO RELATED APPLICATIONS

This application is a continuation of U.S. application Ser. No. 17/330, 115, filed May 25, 2021, which claims the benefit to U.S. Provisional Application No. 63/165,252, filed Mar. 24, 2021, and U.S. Provisional Application No. 63/158,918, filed Mar. 10, 2021, the entire contents of which are incorporated herein by reference in their entirety.

### BACKGROUND

Some control systems seek to minimize a cost function. An example of such a control system is a control device for a medicament delivery device, such as an automatic insulin delivery (AID) device. The cost function for an AID device typically weighs the risk of under-delivery or over-delivery of insulin versus the risk of glucose excursions under or over a control target. In some AID devices, the cost function sums a glucose cost component and an insulin cost component. The glucose component captures the magnitude of glucose excursions above and/or below the control target that are predicted with a candidate dosage, and the insulin component captures the magnitude of insulin above or below a standard dosage (such as a basal dosage) that would be delivered with the candidate dosage. The control system applies the cost function to each candidate dosage of insulin and chooses the candidate dosage with the minimum cost.

AID systems assume that the basal dosage will maintain the blood glucose concentration at a target blood glucose concentration. Unfortunately, this assumption does not hold true for many users. The formulation for the standard basal dosage (e.g., basal dosage calculated from total daily insulin (TDI)) does not match the true needs of many users. For example, suppose that a user needs more than a standard basal dosage of insulin. Candidate dosages above the standard basal dosage are punished by the insulin cost component of the cost function. The insulin cost component is a quadratic expression in the formulation of the cost function. Hence, the magnitude of the insulin cost component escalates rapidly as the candidate dosages increase above the standard basal dosage. This makes it difficult to compensate for lower magnitude glucose excursions because the cost for suitable candidate dosages to rectify the lower magnitude glucose gets high rapidly due to the rapidly escalating insulin cost component. The system thus prefers smaller changes to insulin dosages rather than larger changes, so it may take a long period of time to compensate for such lower magnitude glucose excursions. As such, the user may have persistent low magnitude glucose excursions, which may not be healthy for the patient. The result is that the user may have a persistently higher or lower than target blood glucose concentration.

Another difficulty with the standard formulation of the cost function for AID systems is that there is a penalty for decreasing insulin delivery since there is a delta relative to the basal dosage. This is problematic in instances where the dosage should be quickly decreased to avoid the risk of the user going into hypoglycemia.

### SUMMARY

In accordance with a first inventive aspect, a medicament delivery device includes a memory for storing data and computer programming instructions and a pump for delivering a medicament to a user. The medicament delivery device further includes a processor for executing the computer programming instructions to determine values of a cost function for candidate dosages to the user. The cost function has a performance cost component (e.g., for glucose excursions from a desired target) and a medicament cost component. The medicament cost component is configured to be asymmetrical about a threshold, standard basal, or a customized amount configured to the user. The processor also is configured for executing the computer programming instructions to choose a dosage to be delivered to the user by the pump from among the candidate dosages based on values of the cost function for the candidate dosages.

The threshold amount may be an average basal dosage or a particular basal dosage for the user. The threshold amount may be a multiple of a basal dosage amount for the user, and the multiple is greater than one. The multiple may be a ratio of mean blood glucose concentration over a time interval to target blood glucose concentration. The multiple may be a ratio of average basal dosage delivered to the user over an interval to an estimate of basal dosage over the interval derived from total daily medicament for the user. The choosing of the dosage may comprise choosing one of the candidate dosages with the lowest value for the cost function. The medicament delivery device may deliver at least one of insulin, a glucagon-like peptide (GLP-1) agonist, pramlintide, co-formulations thereof, or another type of drug. The medicament cost component may be zero or substantially zero for any of the candidate dosages below the threshold amount.

In accordance with another inventive aspect, a medicament delivery device includes a memory for storing data and computer programming instructions and a pump for delivering a medicament to a user. The medicament delivery device includes a processor for executing the computer programming instructions to determine values of a cost function for candidate dosages of the medicament for the user and to choose as a dosage to be delivered to the user by the pump among the candidate dosages based on values of the cost function for the candidate dosages. The cost function has a performance cost component and a medicament cost component. The scaling of the medicament cost component is quadratic above a first threshold and linear above a second threshold that is greater than the first threshold.

The scaling of the medicament cost component may be linear below the first threshold. The medicament cost component may have a fixed value for at least one of the candidate dosages that is below the first threshold. The fixed value may be zero or substantially zero.

In accordance with a further inventive aspect, a medicament delivery device includes a memory for storing data and computer programming instructions and a pump for delivering a medicament to a user. The medicament delivery device also includes a processor for executing the computer programming instructions to determine values of a cost for candidate dosages to the user and to choose as a dosage to be delivered to the user by the pump among the candidate dosages to the user based on the values of the cost for the candidate dosages. The cost has a performance cost component and a medicament cost component. The cost is calculated in a different manner for different ranges of the candidate dosages.

The cost may be calculated to be negligible for any one of the candidate dosages in one of the ranges below a first threshold. The first threshold may be an average basal dosage or a particular basal dosage for the user. The first threshold may be a multiple of the average basal dosage or the particular basal dosage for the user, and the multiple may be greater than one. The cost may include a medicament cost component that is calculated by a quadratic formulation in a one of the ranges above a second threshold that is greater than the first threshold. The cost may include a medicament cost component that is calculated by a linear formulation in a one of the ranges above a third threshold that is greater than the second threshold. The cost may be calculated using a cost function, and the cost function may scale differently in at least two of the ranges. The cost function may include a performance cost component and a medicament cost component, and the medicament cost component may differ in the at least two of the ranges to cause the cost function to scale differently in the at least two of the ranges. The cost function may have a medicament cost component, and the cost may be determined for a first of the ranges using a different formula for medicament cost than used in determining the cost for a second of the ranges. The cost may be determined by a different cost function for each range. The medicament may be one of insulin, a glucagon-like peptide-1 (GLP-1) agonist, pramlintide, co-formulations thereof, or another type of drug.

### BRIEF DESCRIPTION OF THE DRAWINGS

FIG. 1 depicts an illustrative medicament delivery system that is suitable for delivering a medicament in accordance with exemplary embodiments.

FIG. 2 depicts a flowchart of illustrative steps that may be performed in choosing a next dosage to be delivered to the user in exemplary embodiments.

FIG. 3 depicts a flowchart of illustrative steps that may be performed to calculate a conventional cost function.

FIG. 4 depicts a plot of the value of the medicament cost component of a cost function versus requested dosage of medicament for a conventional linear medicament cost component and for a conventional quadratic medicament cost component.

FIG. 5 depicts a flowchart of illustrative steps that may be performed in exemplary embodiments to realize the zero cost with the cost function.

FIG. 6 depicts a flowchart of illustrative steps that may be performed to determine a threshold for a medicament cost component based on average blood glucose concentration of a user in exemplary embodiments.

FIG. 7 depicts a flowchart of illustrative steps that may be performed to determine a threshold for a medicament cost component based on an actual ratio of average basal dosage for a user to total daily insulin (TDI) for the user in exemplary embodiments.

FIG. 8A depicts a flowchart of illustrative steps that may be performed to provide mixed scaling across ranges of candidate medicament dosages in exemplary embodiments.

FIG. 8B depicts a plot illustrating mixed scaling across ranges of candidate medicament dosages for a medicament cost component in exemplary embodiments.

FIG. 9 depicts a flowchart of illustrative steps that may be performed as to provide asymmetry in a medicament cost component over ranges of candidate dosage in exemplary embodiments.

### DETAILED DESCRIPTION

The exemplary embodiments concern medicament delivery devices that use cost functions in their control systems to determine medicament dosages. The cost function may have a medicament cost component and a performance cost component. The exemplary embodiments may use cost functions having medicament cost components that scale asymmetrically for different ranges of inputs (i.e., different candidate medicament dosages). The variance in scaling for different input ranges provides added flexibility to tailor the medicament cost component to the user and thus provide better management of medicament delivery to the user and better conformance to a performance target.

The medicament delivery devices of the exemplary embodiments may deliver any of a wide variety of medicaments. The medicaments delivered by the medicament delivery devices of the exemplary embodiments may include but are not limited to insulin, glucagon-like peptide-1 (GLP-1) agonists, pramlintide, co-formulations of two or more of the foregoing, glucagon, hormonal agents, pain management agents, chemotherapy agents, antibiotic agents, anti-viral agents, blood thinning agents, blood clotting agents, anti-depressive agents, anti-seizure agents, anti-psychotic agents, blood pressure reducing agents, statins, therapeutic agents and pharmaceutical agents.

The exemplary embodiments may provide modifications to the cost function of a medicament delivery device relative to conventional medicament delivery devices. For example, the exemplary embodiments may use a cost function that has a medicament cost component (such as an insulin cost component) of zero for candidate dosages for a range of candidate dosages (e.g., below a reference dosage). Alternatively, the medicament cost component may have a negligible value, such as one that is substantially zero, for a range of candidate dosages. For an insulin or GLP-1 agonists delivery device, this reduces the medicament cost component so that there is a reduced penalty for decreasing medicament dosage and makes it easier to avoid hypoglycemia in some instances. This modification to the medicament cost component is configured to reflect the view that the consequences of hypoglycemia are generally more dangerous than hyperglycemia.

In other exemplary embodiments, the medicament cost component may have a fixed positive value (i.e., is a constant value) for candidate dosages in a range. In still other exemplary embodiments, the medicament cost component may be a linear expression in the formulation of the cost function for candidate dosages in a range of candidate dosages. The net effect of these changes to the conventional medicament cost component is to decrease the penalty resulting from the medicament cost component for candidate dosages below the standard dosage relative to the conventional medicament cost component.

The cost function need not use a singular expression or a single type of expression for the medicament cost component over the range of all possible candidate dosages. The medicament cost expression may be a constant, a linear expression, a quadratic expression or an exponential expression that is not quadratic. The medicament cost component may have different expressions over ranges of candidate dosages. For example, a medicament cost component may be a constant for a first range of candidate dosages, a non-constant linear expression for a second range of candidate dosages and a quadratic expression for a third range of candidate dosages. In some embodiments, the medicament cost component expression may be of a single type (e.g., constant, linear, quadratic or exponential) over the range of candidate dosages but may have a different formulation. For example, the medicament cost component may be expressed as x+2 for a first range and 2x+3 for a different range, where x is a variable such as a delta relative to a basal dosage.

In some exemplary embodiments, different formulations of the cost function may be used for different ranges or even different cost functions may be used for different ranges. The differences need not be due solely to changes to the medicament cost component. The scaling of the medicament cost component need not be the same over all possible candidate dosages. The cost function may be asymmetric across a threshold or reference dosage.

The exemplary embodiments may also change the reference dosage that is used in determining the medicament cost component. First, the reference dosage may be set higher than a basal dosage. Thus, the reference dosage may be better suited for a user with higher than normal medicament needs, such as higher insulin needs. Second, the reference dosage may be customized to a user's actual average basal amount (such as an average over a recent interval). This tailors the reference dosage value better to the user. Third, the reference dosage may be customized based on a user's recent actual split between basal delivery of medicament and bolus delivery of medicament. The reference dosage is not limited to being 50% of TDI in the cost calculation.

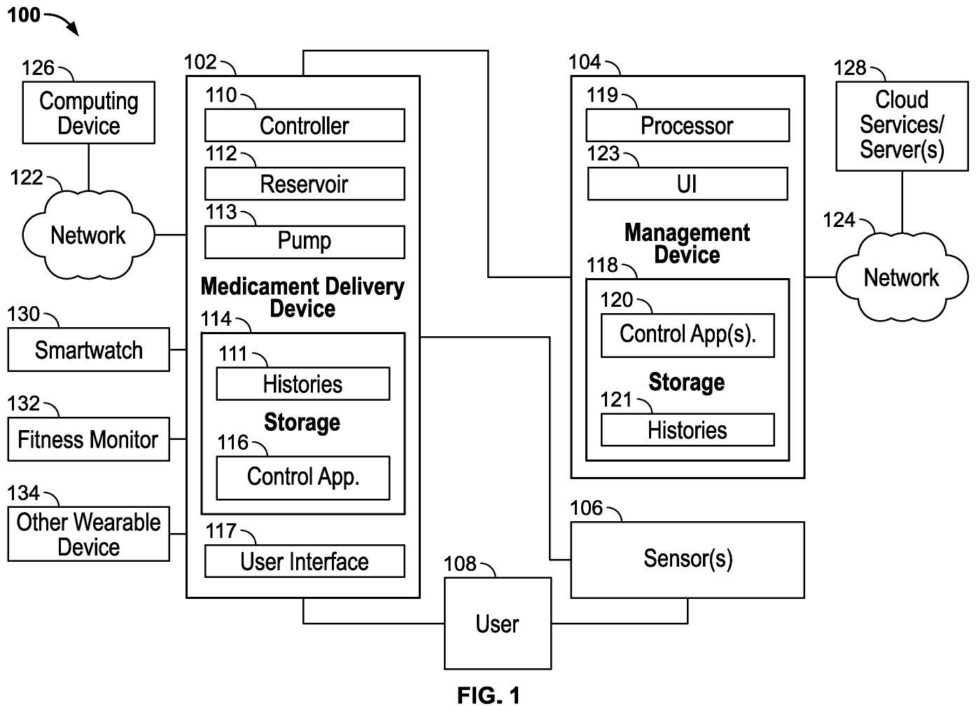

FIG. 1 depicts an illustrative medicament delivery system 100 that is suitable for delivering a medicament, such as insulin, a GLP-1 agonist or other medicament like those detailed above, to a user 108 in accordance with exemplary embodiments. The medicament delivery system 100 includes a medicament delivery device 102. The medicament delivery device 102 may be a wearable device that is worn on the body of the user 108. The medicament delivery device 102 may be directly coupled to a user (e.g., directly attached to a body part and/or skin of the user 108 via an adhesive or the like). In an example, a surface of the medicament delivery device 102 may include an adhesive to facilitate attachment to the user 108.

The medicament delivery device 102 may include a controller 110. The controller 110 may be implemented in hardware, software, or any combination thereof. The controller 110 may, for example, be a microprocessor, a logic circuit, a field programmable gate array (FPGA), an application specific integrated circuit (ASIC) or a microcontroller coupled to a memory. The controller 110 may maintain a date and time as well as other functions (e.g., calculations or the like). The controller 110 may be operable to execute a control application 116 stored in the storage 114 that enables the controller 110 to implement a control system for controlling operation of the medicament delivery device 102. The control application 116 may control medicament delivery to the user 108 as described herein. The storage 114 may hold histories 111 for a user, such as a history of automated medicament deliveries, a history of bolus medicament deliveries, meal event history, exercise event history, sensor data and the like. In addition, the controller 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include a reservoir 112 for storing medicament for delivery to the user 108 as warranted. A fluid path to the user 108 may be provided, and the medicament delivery device 102 may expel the medicament from the reservoir 112 to deliver the medicament to the user 108 via the fluid path. The fluid path may, for example, include tubing coupling the medicament delivery device 102 to the user 108 (e.g., tubing coupling a cannula to the reservoir 112).

There may be one or more communications links with one or more devices physically separated from the medicament delivery device 102 including, for example, a management device 104 of the user and/or a caregiver of the user and/or a sensor 106. The communication links may include any wired or wireless communication link operating according to any known communications protocol or standard, such as Bluetooth®, Wi-Fi, a near-field communication standard, a cellular standard, or any other wireless protocol The medicament delivery device 102 may also include a user interface 117, such as an integrated display device for displaying information to the user 108 and in some embodiments, receiving information from the user 108. The user interface 117 may include a touchscreen and/or one or more input devices, such as buttons, a knob or a keyboard.

The medicament delivery device 102 may interface with a network 122. The network 122 may include a local area network (LAN), a wide area network (WAN) or a combination therein. A computing device 126 may be interfaced with the network, and the computing device may communicate with the insulin delivery device 102.

The medicament delivery system 100 may include sensor(s) 106 for sensing the levels of one or more analytes. The sensor(s) 106 may be coupled to the user 108 by, for example, adhesive or the like and may provide information or data on one or more medical conditions and/or physical attributes of the user 108. The sensor(s) 106 may, in some exemplary embodiments, provide periodic blood glucose concentration measurements and may be a continuous glucose monitor (CGM), or another type of device or sensor that provides blood glucose measurements. The sensor(s) 106 may be physically separate from the medicament delivery device 102 or may be an integrated component thereof. The sensor(s) 106 may provide the controller 110 with data indicative of one or more measured or detected analyte levels of the user 108. The information or data provided by the sensor(s) 106 may be used to adjust medicament delivery operations of the medicament delivery device 102.

The medicament delivery system 100 may also include the management device 104. In some embodiments, no management device 104 is needed; rather the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller or the like. The management device 104 may be used to program or adjust operation of the medicament delivery device 102 and/or the sensor 104. The management device 104 may be any portable electronic device including, for example, a dedicated device, a smartphone, a smartwatch or a tablet. In the depicted example, the management device 104 may include a processor 119 and a storage 118. The processor 119 may execute processes to manage and control the delivery of the medicament to the user 108. The processor 119 may also be operable to execute programming code stored in the storage 118. For example, the storage may be operable to store one or more control applications 120 for execution by the processor 119. The one or more control applications 120 (or 116) may be responsible for controlling the medicament delivery device 102, e.g., delivery of insulin to the user 108. The storage 118 may store the one or more control applications 120, histories 121 like those described above for the medicament delivery device 102 and other data and/or programs.

The management device 104 may include a user interface (UI) 123 for

communicating with the user 108. The user interface 123 may include a display, such as a touchscreen, for displaying information. The touchscreen may also be used to receive input when it is a touch screen. The user interface 123 may also include input elements, such as a keyboard, buttons, knob(s), or the like. The user interface 123 may be used to view data or history or provide input, such as to cause a change in basal medicament dosage, deliver a bolus of medicament, or change one or more parameters used by the control app 116/120.

The management device 104 may interface with a network 124, such as a LAN or WAN or combination of such networks. The management device 104 may communicate over network 124 with one or more servers or cloud services 128.

Other devices, like smartwatch 130, fitness monitor 132 and/or another wearable device 134 may be part of the medicament delivery system 100. These devices may communicate with the medicament delivery device 102 to receive information and/or issue commands to the medicament delivery device 102. These devices 130, 132 and 134 may execute computer programming instructions to perform some of the control functions otherwise performed by controller 110 or processor 119. These devices 130, 132 and 134 may include displays for displaying information, e.g., analyte levels like current blood glucose level, medicament on board, medicament delivery history, etc. The display may show a user interface for providing input, such as to cause a change in basal medicament dosage, delivery of a bolus of medicament, or a change of one or more parameters used by the control application 116/120. These devices 130, 132 and 134 may also have wireless communication connections with the sensor 106 to directly receive analyte data.

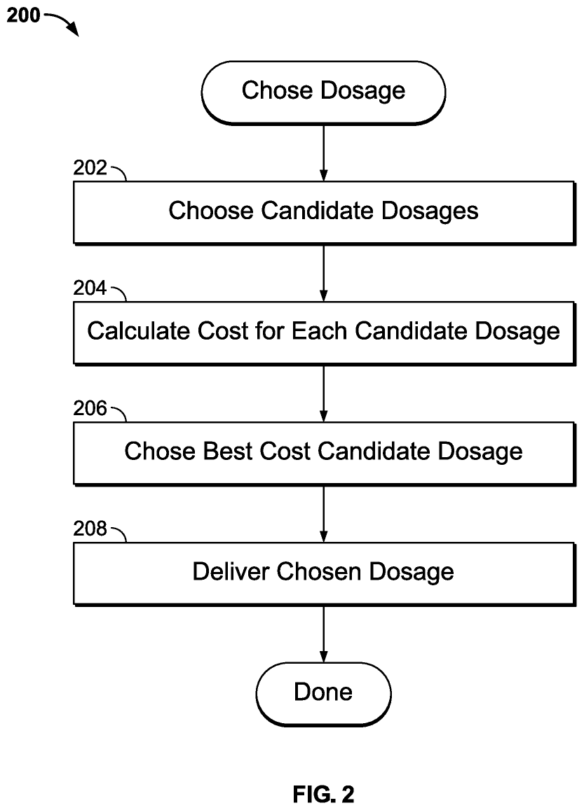

The control system of the exemplary embodiments relies upon a cost function as mentioned above. The control system attempts to minimize the aggregate penalty of the cost function over a wide range of possible candidate medicament dosages. FIG. 2 depicts a flowchart 200 of illustrative steps that may be performed in choosing a next dosage to be delivered to the user in exemplary embodiments. At 202, the control system chooses the candidate dosages from which the dosage may be chosen. At 204, a cost is calculated for each candidate dosage using the cost function. The cost function has the candidate dosage as an input and the cost for the candidate dosage as an output. At 206, the candidate dosage with the best cost is chosen. Depending on how the cost function is configured, the best cost may be the lowest value or the highest value. For discussion purposes hereinafter, it is assumed that the lowest cost candidate dosage is the best cost. At 208, the chosen dosage is delivered to the user. A control signal may be generated and sent from the controller 110 running the control application 116 to the pump 113 to cause the pump 113 to deliver the chosen medicament dosage to the user. Thereafter, process 200 may be repeated for another subsequent cycle (e.g., every 5 minutes).

In order to appreciate how the exemplary embodiments modify a cost function for a medicament delivery device, it is helpful to look at a conventional cost function for a medicament device. An example of a cost function for a medicament delivery device is a cost function for an insulin delivery device. A conventional cost function for an insulin delivery device is:

J=Q·Σᵢ₌₁^{M}Gₚ(i)²+R·Σᵢ₌₁ⁿIₚ(i)² (Equation 1)

where J is the cost, Q and R are weight coefficients, Gₚ(i)² is the square of the deviation between a projected blood glucose concentration for a particular (e.g., candidate) insulin dosage at cycle i and the projected blood glucose concentration for the standard basal insulin dosage, M is the number of cycles in the prediction horizon (a cycle is a fixed interval, such 5 minutes), Iₚ(i)² is the square of the deviation between the projected insulin delivered at cycle i and the standard insulin for basal insulin delivery, and n is the control horizon in cycles.

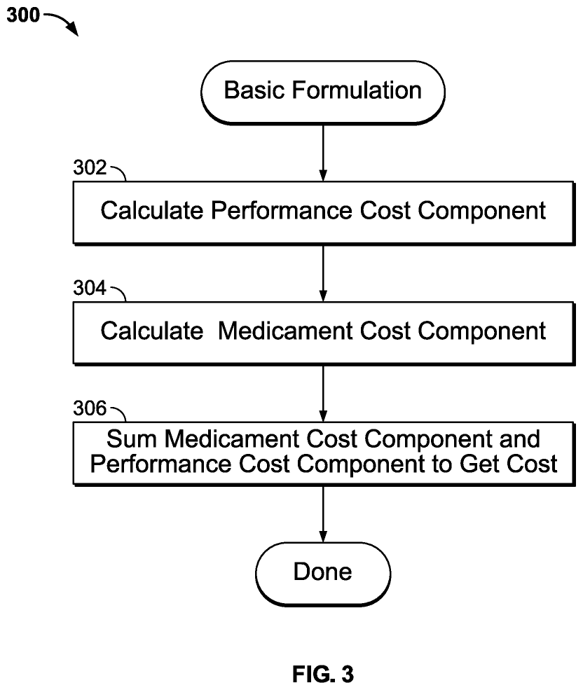

FIG. 3 depicts a flowchart 300 of illustrative steps that may be performed to calculate a cost function, such as that detailed in Equation 1. At 302, the medicament cost component is calculated. For this cost example conventional cost function, the performance cost component is the expression Q·Σᵢ₌₁^{M}Gₚ(i)², which is the glucose cost component. The performance cost component captures the cost in deviation from the target performance. For insulin delivery, the target performance is measured relative to a target blood glucose concentration. At 304, the medicament cost component is calculated. The medicament cost component captures the penalty of deviating from a reference medicament dosage, such as a standard basal dosage. In this conventional cost function, the medicament cost is the expression R·Σᵢ₌₁ⁿIₚ(i)², which is the weighted insulin cost. At 306, the total cost J is calculated as the sum of the performance cost component and the medicament cost component.

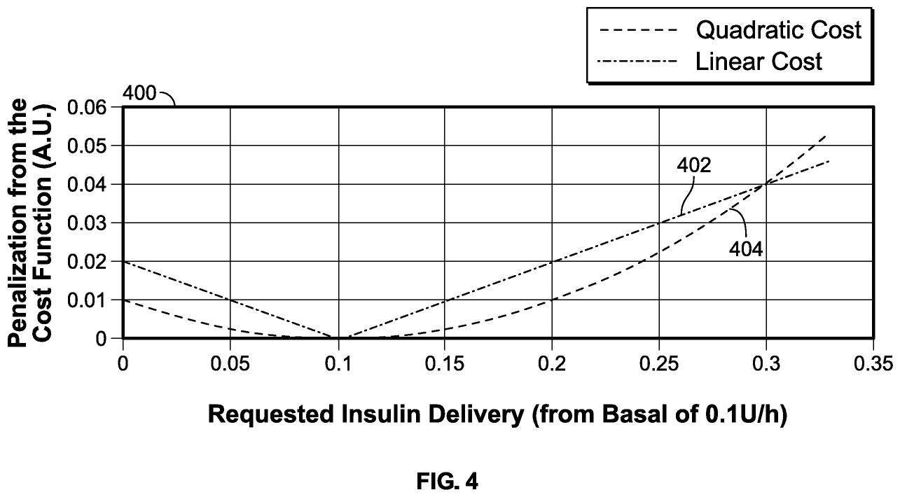

Cost functions for conventional medicament delivery devices tend to specify the medicament cost function as a linear cost or a quadratic cost. In other words, the medicament cost function is expressed as a linear expression or as a quadratic expression. The conventional cost function described above has a quadratic medicament cost component since R·Σᵢ₌₁ⁿIₚ(i)² is a quadratic expression. FIG. 4 depicts a plot 400 of the value of the medicament cost component of a cost function (see y-axis) versus a requested dosage of medicament (i.e., candidate dosage amount) for a linear medicament cost component and for a quadratic medicament cost component. The linear medicament cost component is represented by curve 402, and the quadratic medicament cost component is represented by curve 404 in the plot 400. The linear medicament cost component 402 has a linear formulation that scales in a linear fashion (i.e. along a line). The quadratic medicament cost component 404 has a quadratic formulation that scales quadratically. In the example of FIG. 4, the standard basal dosage is 0.1 units of insulin per hour. With the linear medicament cost component, the curve 402 shows that the penalty or cost decreases linearly from 0.02 (y-axis) when no insulin is requested to a zero penalty or cost for the medicament cost component at a requested dosage equal to the standard basal dosage. The penalty increases linearly for increasing dosages above or below the standard basal dosage such that the cost is symmetric about the standard basal dosage. For the quadratic medicament cost component, the penalty or cost starts at 0.01 (y-axis) when no insulin is requested and decreases in a quadratic fashion (i.e., parabolically) until a dosage equal to the standard basal dosage is reached. The penalty or cost increases in a quadratic fashion for dosages over the standard basal dosage such that the cost is symmetric about the standard basal dosage. The rate of increase of the penalty for the quadratic medicament cost component exceeds that of the linear medicament cost component for higher dosages such that, in this example, starting at a requested dosage of 0.3 U/h, the penalty for requested dosages with the quadratic medicament cost component exceeds that of the linear medicament cost component.

As can be seen from FIG. 4, with both the conventional linear medicament cost component and the conventional quadratic medicament cost component, there is a penalty for delivering insulin dosages below the standard basal dosage. The exemplary embodiments described below and in subsequent figures recognize that such a penalty may not be desirable. As was described above, such a penalty discourages delivering insulin dosages below a standard basal dosage. Thus, in accordance with some exemplary embodiments, the medicament cost component is zero or substantially zero (i.e., slightly more than zero but still negligible) for requested dosages below the standard basal dosage (i.e., the reference dosage mentioned above). This formulation of the medicament cost component allows the control system to be free to reduce medicament delivery as much as is needed to modify the predicted performance metric to the setpoint. This control approach can be more aggressive in reducing medicament delivery, while maintaining compensation for increases in medicament delivery relative to a standard basal dosage.

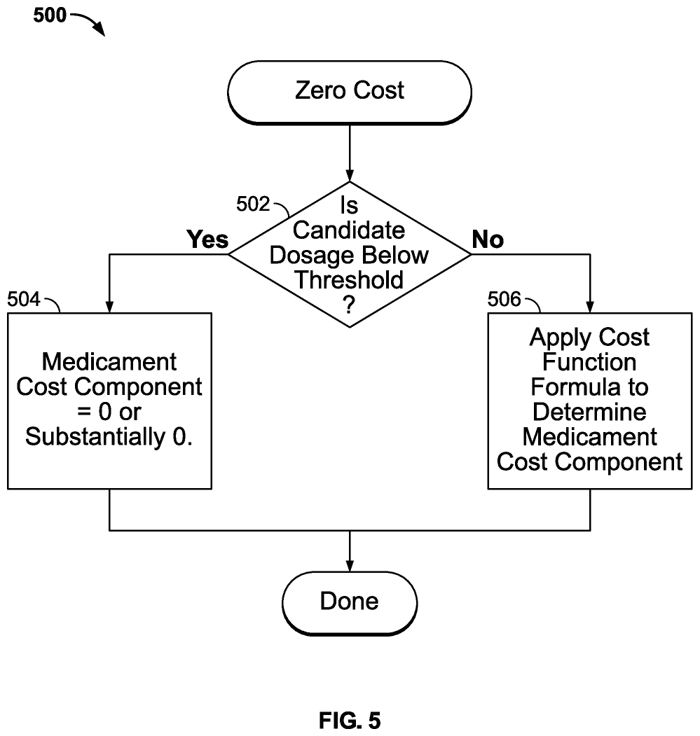

FIG. 5 depicts a flowchart 500 of illustrative steps that may be performed in exemplary embodiments to realize a zero cost with the cost function. At 502, a check is made whether the candidate dosage is below a threshold, such as below a standard basal dosage. At 504, if the candidate dosage is below the threshold, the medicament cost component is set as zero or at a value that is substantially zero. At 506, if the candidate dosage is equal to or above the threshold, the medicament cost component is calculated using a formulation specified in the cost function.

An example formulation of a cost function that provides a zero-cost medicament cost component value below a threshold or standard basal dosage for insulin delivery is:

J=Q·Σᵢ₌₁^{M}Gₚ(i)²+R·Σᵢ₌₁ⁿIₚ(i)² for iᵣ>I_{b} (Equation 2.1)

and

J=Q·Σᵢ₌₁^{M}Gₚ(i)² for Iᵣ≤I_{b} (Equation 2.2)

where Iᵣ is the requested dosage of insulin (i.e., a candidate dosage) and Ibis basal dosage. Values substantially equal to 0 for the medicament cost component may be used instead of 0 in some exemplary embodiments.

The reference value that is used as a threshold below which the medicament cost component is zero or substantially zero may be a value other than the standard basal dosage. Sometimes, a user may have greater medicament needs than the standard basal formulation. As such, pegging deliveries at the standard basal dosage may not get rid of persistent low-level performance excursions above a blood glucose target. Therefore, some exemplary embodiments may set the threshold based on mean positive performance excursions above the blood glucose target. This enables the delivery of larger than standard basal dosages of medicament to reduce the positive performance excursions without penalty.

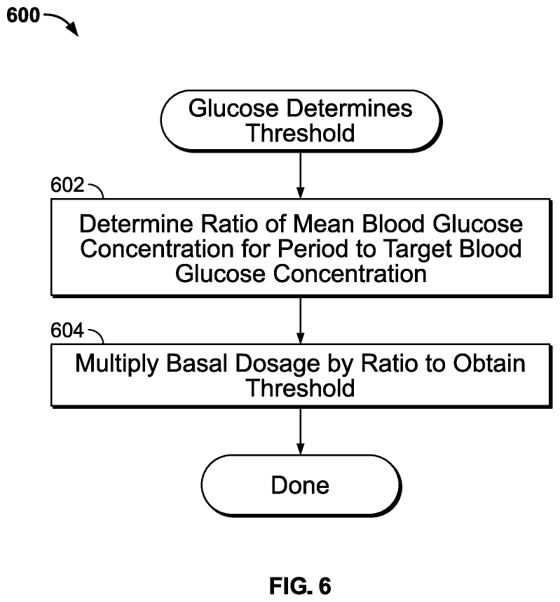

One application of the threshold being adaptable higher than a standard basal dosage is for an insulin delivery device. In such an application, the threshold may be calculated as shown in the flowchart 600 of FIG. 6. At 602, a ratio of mean blood glucose concentration of a user for a period to target blood glucose concentration is calculated. This ratio reflects the degree to which a user's mean blood glucose concentration exceeds the target blood glucose concentration. At 604, the basal dosage is multiplied by the ratio to determine the threshold.

The cost function with the modified threshold base on mean glucose excursions may be expressed as:

$$J = Q \cdot {\sum }_{i = 1}^{M} {( G ( i ) - SP )}^{2} + R \cdot {I}_{p} ( i ) \qquad ( Equation 3.1 )$$

$${I}_{p} ( i ) = {\sum }_{i = 1}^{n} {( {I}_{r}^{+} ( i ) - {I}_{b} ( i ) )}^{2} \qquad ( Equation 3.2 )$$

$${I}_{r}^{+} ( i ) = \{ \begin{matrix}{I}_{r} & {I}_{r} \geq \frac{{G}_{mean}}{{G}_{t}} \cdot {I}_{b} \\ {I}_{b} & {I}_{r} < \frac{{G}_{mean}}{{G}_{t}} \cdot {I}_{b}\end{matrix} \qquad ( Equation 3.4 )$$

$${G}_{mean} = \frac{{\sum }_{j = 1}^{12 X} G ( j )}{12 X} \qquad ( Equation 3.4 )$$

where Gₜ the mean blood glucose concentration target. The threshold is

$$\frac{{G}_{mean}}{{G}_{t}} \cdot {I}_{b} .$$

Since the value of Iᵣ⁺(i) is I_{b} for values below the threshold, the insulin cost component R·Iₚ(i) is 0, since Iₚ(i) is I_{b}-I_{b} or 0.

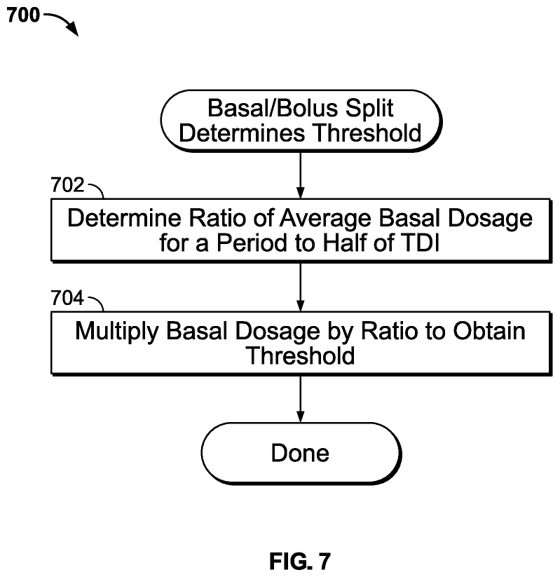

The threshold instead may be set based on a reference value of a user's actual basal/TDI split. Traditionally, the basal amount for a user is set at half of the user's TDI. Unfortunately, this rule of thumb does not work well for some users. To account for such users, the exemplary embodiments may determine the threshold based on the user's actual basal/TDI ratio. FIG. 7 depicts a flowchart 700 of steps that may be performed to determine such a threshold. At 702, the ratio of average basal dosage for a user over a period (e.g., daily) to half of the TDI for the user is determined. If the user has higher than normal insulin needs (i.e. greater than basal), the ratio will be greater than one. At 704, the standard basal dosage (i.e., 0.5 TDI) for the user is multiplied by the ratio to determine the threshold reference value. This enables the threshold to exceed the basal dosage where the average actual basal dosage is higher than the standard basal dosage.

The formulation of the cost function may be as described for the mean glucose excursions as described above, but Iᵣ⁺(i) may be differently formulated as follows:

$$\\ \begin{matrix}{I}_{r}^{{}_{} +} ( i ) = \{ \begin{matrix}{I}_{r} & {I}_{r} \geq max ( 1 , \frac{{I}_{btotal} ( t )}{0.5 TDI} ) \cdot {I}_{b} \\ {I}_{b} & {I}_{r} < max ( 1 , \frac{{I}_{btotal} ( t )}{0.5 TDI} ) \cdot {I}_{b}\end{matrix} & ( Equation 4.1 )\end{matrix}$$

$$\\ \begin{matrix}{I}_{btotal} ( t ) = 288 \frac{{\sum }_{j = 1}^{12 X} {I}_{bactual} ( j )}{12 X} & ( Equation 4.2 )\end{matrix}$$

where I_{btotal} is the total basal insulin over an interval and I_{bactual}(i) is the actual basal insulin at cycle i.

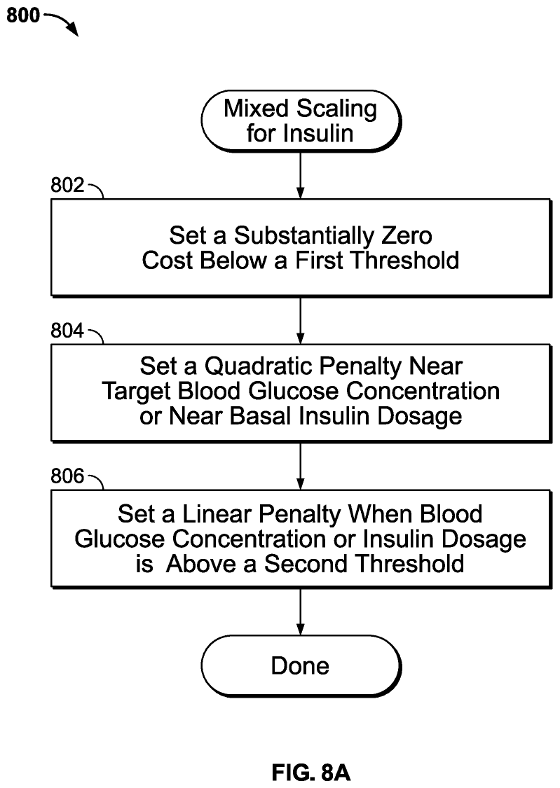

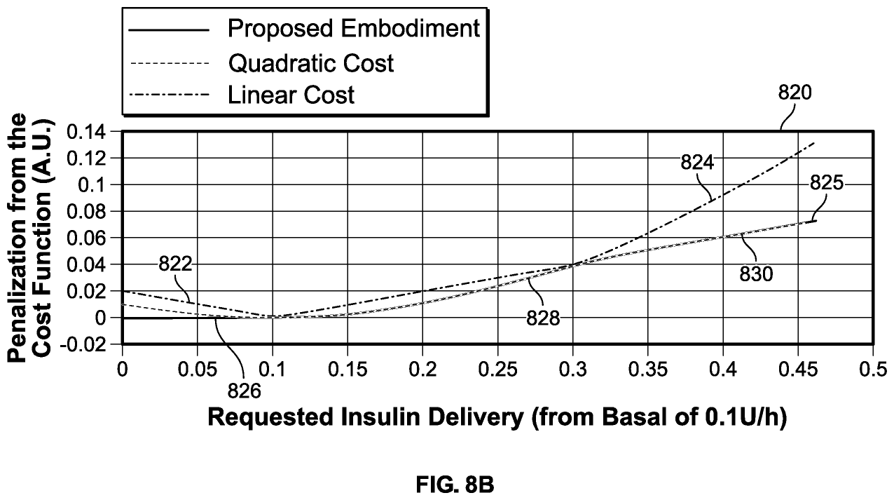

It may be desirable for a medicament cost component in a cost function to be asymmetric among different ranges to suit the medicament needs of the user. In other words, the medicament cost component may be differently scaled in different ranges. FIG. 8A depicts a flowchart 800 of an example where different scaling is used for different ranges of the medicament candidate dosages. In this example, at 802, the medicament cost component has a zero cost below a first threshold. An illustration of this is seen in plot 820 of FIG. 8B. The plot 820 shows curve 822, which has linear scaling, and curve 824 which has quadratic scaling like that depicted in FIG. 4. Curve 825 represents the medicament cost component over a range of candidate dosages, with scaling changing at different basal dosage rates. The region 826 of the curve before a requested insulin delivery of 0.1 U/h has a flat constant value of 0, such as was discussed above relative to FIG. 5. At 804, the scaling of the curve 825 changes to a quadratic scaling in region 828 that extends from 0.1 U/h to 0.3 U/h. This region 828 represents the range of requested dosages when the blood glucose concentration of the user is near target and the requested insulin dosage is slightly over the basal dosage. The quadratic scaling is well suited for this region 828, where insulin levels are elevated but not so large as to pose a threat to causing hypoglycemia. However, as the candidate dosage exceeds 0.3 U/h, the quadratic scaling may be too aggressive and may increase the risk of hypoglycemia. Thus, at 806, the scaling is reduced. Specifically, the insulin cost component has linear scaling when the requested insulin dosage is above a second threshold (e.g., 0.3 U/h) in region 830. This reduces the risk of hypoglycemia due to delivering too large of dosages of insulin.

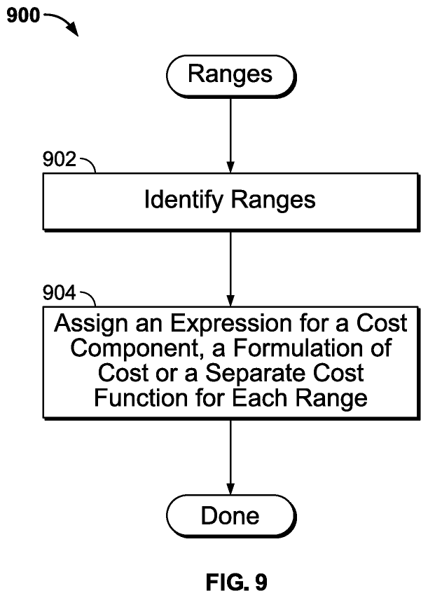

The above-described examples show that the medicament cost component and the cost function may be varied over different ranges of inputs (i.e., medicament dosages). It should be noted that more generally, the exemplary embodiments may use cost functions with asymmetry for the medicament cost components. FIG. 9 depicts a flowchart 900 of illustrative steps that may be performed as to provide asymmetry over the ranges. At 902, the ranges of candidate dosages of medicament are identified. At 904, an expression is assigned for a medicament cost component for each range, a formulation of the cost for each range is assigned, or a separate cost function is assigned for each range. This assignment provides the asymmetry over the ranges of candidate dosages. This asymmetry enables the control system to better customize the medicament cost components to a user's needs.

While the discussion herein has focused on exemplary embodiments, it should be appreciated that various change in form and detail may be made relative to the exemplary embodiments without departed from the intended scope of the claims appended hereto.

1. A system, comprising:
a memory for storing data and computer programming instructions;
a processor for executing the computer programming instructions to:
determine values of a cost function for candidate doses of a medicament that affects a glucose level of a user;
wherein the cost function has a performance cost component and a medicament cost component;
wherein the candidate doses are selected from a range of doses that includes subranges of doses;
wherein the medicament cost component is configured to be calculated in a different manner for candidates doses in a first of the subranges versus candidate doses in a second of the subranges; and
choose a dose of the medicament to be delivered to the user from among the candidate dosages based on values of the cost function for the candidate doses.

2. The system of claim 1, wherein one of the first subrange or the second subrange includes doses that are greater than an average basal dose for the user.

3. The system of claim 1, wherein one of the subranges includes doses that are less than an average basal dose for the user.

4. The medicament delivery device of claim 1, wherein the choosing of the dose comprises choosing one of the candidate doses with a lowest value for the cost function.

5. The system of claim 1, wherein the medicament includes at least one of insulin, a glucagon-like peptide (GLP-1) agonist, or pramlintide.

6. The system of claim 1, wherein the medicament cost component is substantially zero for doses in one of the first subrange or the second subrange.

7. The system of claim 1, wherein the first subrange includes doses that are greater than a standard insulin dose for the user and the second subrange includes doses that are less than the standard insulin dose.

8. The system of claim 7, wherein if a candidate dose is in the second subrange, the medicament cost component is negligible or zero.

9. A medicament delivery system, comprising:
a memory for storing data and computer programming instructions;
a pump for delivering a medicament to a user;
a processor for executing the computer programming instructions to:
determine values of a cost function for candidate dosages of the medicament to the user, the cost function has a performance cost component and a medicament cost component,
wherein scaling of the medicament cost component differs above a first threshold from scaling of the medicament cost component above a second threshold, which is greater than the first threshold; and
choose a dosage to be delivered by the pump among the candidate dosages based on values of the cost function for the candidate dosages.

10. The medicament delivery system of claim 9, wherein the scaling of the medicament cost component is linear below the first threshold.

11. The medicament delivery system of claim 10, wherein the medicament cost component has a fixed value for at least one of the candidate dosages that is below the first threshold.

12. The medicament delivery system of claim 11, wherein the fixed value is zero or substantially zero.

13. A medicament delivery system, comprising:
a memory for storing data and computer programming instructions;
a medicament delivery component;
a processor for executing the computer programming instructions to:
determine values of a cost for candidate dosages of the medicament, the cost has a performance cost component and a medicament cost component;
wherein the values of the cost are calculated in a different manner for different ranges of the candidate dosages; and
choose as a dosage to be delivered to a user by the medicament delivery component among the candidate dosages to a user based on values of the cost for the candidate dosages.

14. The medicament delivery system of claim 13, wherein the values of the cost are calculated to be negligible for ones of the candidate dosages below a first threshold.

15. The medicament delivery system of claim 14, wherein the first threshold is a standard basal dosage for the user.

16. The medicament delivery system of claim 15, wherein the first threshold is a multiple of the standard basal dosage for the user and the multiple is greater than one.

17. The insulin delivery system of claim 13, wherein the values of the cost are determined by a cost function for which the medicament cost component scales differently in at least two of the ranges.

18. The insulin delivery system of claim 17, wherein the values of the cost are determined for a first of the ranges using a different formula for the medicament cost component than used in determining the values of the cost for a second of the ranges.

19. The insulin delivery system of claim 13, wherein the value of the cost is determined by a different cost function for each of the ranges.

20. The medicament delivery system of claim 13, wherein the medicament is one of insulin, a glucagon-like peptide-1 (GLP-1) agonist, or pramlintide.
